(19) United States
(12) Patent Application Publication
(10) Pub. No.: US 2025/0268579 A1
(43) Pub. Date: Aug. 28, 2025

(54) RINGDOWN ARTIFACT SUPPRESSION FOR ULTRASOUND-BASED DETECTION

(71) Applicant: University of Washington, Seattle, WA (US)

(72) Inventors: Yana Sosnovskaya, Seattle, WA (US); Blake Hannaford, Seattle, WA (US); Eli Shlizerman, Seattle, WA (US); Mika N. Sinanan, Seattle, WA (US)

(73) Assignee: University of Washington, Seattle, WA (US)

(21) Appl. No.: 19/064,433

(22) Filed: Feb. 26, 2025

Related U.S. Application Data
(60) Provisional application No. 63/559,121, filed on Feb. 28, 2024.

(51) Int. Cl. A61B 8/00; A61B 17/00; A61B 17/29

(52) U.S. Cl. CPC A61B 8/5269; A61B 17/29; A61B 2017/00106

(57) ABSTRACT

An example method includes transmitting, by a single ultrasound transducer, an incident ultrasound signal and detecting, at least partially from an object within a ringdown range of the single ultrasound transducer and by the single ultrasound transducer, a received ultrasound signal. A ringdown artifact is removed from data indicative of the received ultrasound signal. Based on removing the ringdown artifact, the object is analyzed based on the data.

### CROSS-REFERENCE TO RELATED APPLICATION

This application claims priority to U.S. Provisional App. No. 63/559,121, which was filed on Feb. 28, 2024, and is incorporated by reference herein in its entirety.

### STATEMENT REGARDING FEDERALLY SPONSORED RESEARCH OR DEVELOPMENT

This invention was made with government support under Grant No. 2036255, awarded by the National Science Foundation. The government has certain rights in the invention.

### BACKGROUND

Minimally Invasive Surgery (MIS) involves operating through small incisions using laparoscopic instruments (e.g., graspers) for manipulation, and endoscopic cameras for visual feedback. Advantages of MIS include faster recovery, less blood loss, and a lower risk of complications. Alongside its benefits, MIS also brings new challenges, such as the lack of tactile feedback for surgeons (Nagy et al., Proc. SACI, pp. 99-106, 2019; Schostek et al., Med. Eng. Phys., vol. 31, no. 8, pp. 887-898, 2009). In open surgery, surgeons can palpate the tissue to gain information about its non-visible structure, such as the location of tumors and blood vessels (Nagy et al., Proc. SACI, pp. 99-106, 2019).

Adding miniaturized sensors to conventional laparoscopic graspers provide tactile feedback to surgeons. Such devices paired with artificial intelligence (AI) algorithms for noise removal and data processing can provide valuable new information to the surgeons, and help with early diagnosis and treatment. Previously, a sensorized laparoscopic grasper was developed (Roan et al., Appl. Bionics Biomech., vol. 8, pp. 173-90, 2011), in which a combination of multisensory data from the grasper and a machine learning algorithm were used to detect tissue ischemia. Another laparoscopic grasper used an artificial neural network (ANN) and sensors to compensate its force measurements for the environmental factors (Seok et al., IEEE Robot. Automat. Lett., vol. 4, no. 2, pp. 2031-37, 2019).

Ultrasound is widely used for diagnostic, prognostic and treatment applications, such as abnormality detection, tissue characterization, high-intensity ultrasound ablation therapy and others. An example ultrasound transducer includes a piezoelectric crystal that has a resonant oscillation frequency. Piezoelectric crystals can operate as both transmitters and receivers for sound waves. If a time-varying potential difference is applied across the electrodes, a piezoelectric crystal may oscillate and produce a sound wave. Conversely, if a sound wave is applied to the piezoelectric crystal, it will generate a voltage across the electrodes. When an ultrasound pulse travels through the tissue, it undergoes continuous modifications, which depend on the characteristics of the sound waves as well as tissue properties. Several characteristics of these sound waves are particularly important for tissue characterization, such as time-of-flight (TOF), which depends on the velocity of sound, and attenuation (P. Fish, Physics and Instrumentation of Diagnostic Medical Ultrasound. New York, NY, USA: John Wiley & Sons, 1990).

### BRIEF DESCRIPTION OF THE DRAWINGS

FIG. 1 illustrates an example surgical environment for detecting tissue characteristics using a surgical instrument.

FIGS. 2A to 2B illustrate raw ultrasound signals with ringdown artifacts. FIG. 2A illustrates a long-distance ultrasound signal with artifact. FIG. 2B illustrates a short distance ultrasound signal with artifact.

FIG. 3 illustrates a generic environment for enhancing the detection range of an ultrasound transducer by suppressing a ringdown artifact.

FIG. 4 illustrates an example process for enhancing ultrasound detection of an object using ringdown artifact suppression.

FIG. 5 illustrates at least one device configured to execute various functions described herein.

FIG. 6 illustrates an example of a surgical grasper with an attached ultrasound transducer on the tip.

FIG. 7 illustrates an example of an architecture for ringdown artifact suppression and time of flight (TOF) estimation.

FIG. 8 illustrates an example of an acrylic container with an attached ultrasound transducer at the bottom.

FIG. 9 illustrates example denoising results for a bandpass filter, a adaptive least mean squares (LMS) filter, spectrum suppression (SPS), gated recurrent unit (GRU), long short-term memory (LSTM), and recurrent neural network (RNN) techniques. The left side of FIG. 9 illustrates example results of the denoising techniques in the time domain. The vertical lines show the true TOF with the travel distance of 1 cm. The right side of FIG. 9 illustrates example results of the denoising techniques in the frequency domain.

FIG. 10 illustrates example noise removal signal-to-noise ratio (SNR) results for the bandpass filter, the adaptive LMS Filter, SPS, GRU, LSTM, and RNN techniques.

FIGS. 11A to 11D illustrate example TOF mean percentage errors by different filtering and TOF estimation methods. FIG. 11A illustrates example TOF errors using a minimum threshold estimation method. FIG. 11B illustrates example TOF errors with a Hilbert envelope estimation method. FIG. 11C illustrates example TOF errors with a cross-correlation estimation method. FIG. 11D illustrates example TOF errors with a short-time Fourier transform (STFT) estimation method.

FIG. 12 illustrates example relative TOF errors for filtering and TOF estimation method pairs across various distances.

FIG. 13 illustrates example Wilcoxon p-values for RNN vs spectrum suppression, RNN vs bandpass filter, and RNN vs adaptive LMS filter using the cross-correlation TOF estimation method.

### DETAILED DESCRIPTION

A significant limitation of processing signals detected by existing ultrasound transducers is the existence of the ringdown artifact, which can limit the range of locations that can be monitored by the transducers. When an ultrasound transducer operates as both a transmitter of ultrasound and a receiver of reflected ultrasound, a prolonged ringing of the piezoelectric crystal after transmission can interfere in the detection of the reflected ultrasound. The ringdown artifact caused by the ringing may be embodied as an artifact in the voltage detected from the transducer. The ringdown artifact, for instance, can overlap reflections of ultrasound detected from objects within a relatively close range (e.g., a couple of centimeters (cm) or less) from the transducer. Accordingly, the transducer may be prevented from identifying the position or characteristics of objects within the relatively close range of the transducer. The ringdown artifact, for instance, can be particularly problematic in applications of ultrasound to MIS and other surgical techniques, where it may be desirable to monitor the positions and characteristics of tissue structures within close proximity to the transducer.

According to various implementations of the present disclosure, these and other problems can be addressed by suppressing the ringdown artifact. For example, a transducer is configured to detect an ultrasound signal during a time period that overlaps with ringing of a piezoelectric crystal of the transducer. One or more techniques described herein can be utilized to suppress or substantially eliminate the ringdown artifact from data representing the voltage across the piezoelectric crystal over time. For instance, in some cases, a recurrent neural network (RNN) is trained to remove the ringdown artifact. Once the ringdown artifact is suppressed, the data can be analyzed in order to determine a feature of an object or material from which the ultrasound signal has been reflected. For example, in various techniques in which the transducer itself transmits the ultrasound signal and detects the reflection of the ultrasound signal from the object, the time-of-flight of the ultrasound signal can be accurately estimated by analyzing the data. Accordingly, the location of the object can be accurately estimated.

Although various implementations described herein relate to suppressing a ringdown artifact of an ultrasound signal, implementation of the present disclosure are not limited to ultrasound signals. In some aspects, similar techniques can be utilized to suppress a ringdown artifact of a radar or sonar signal.

Various implementations of the present disclosure will now be described with reference to the accompanying figures.

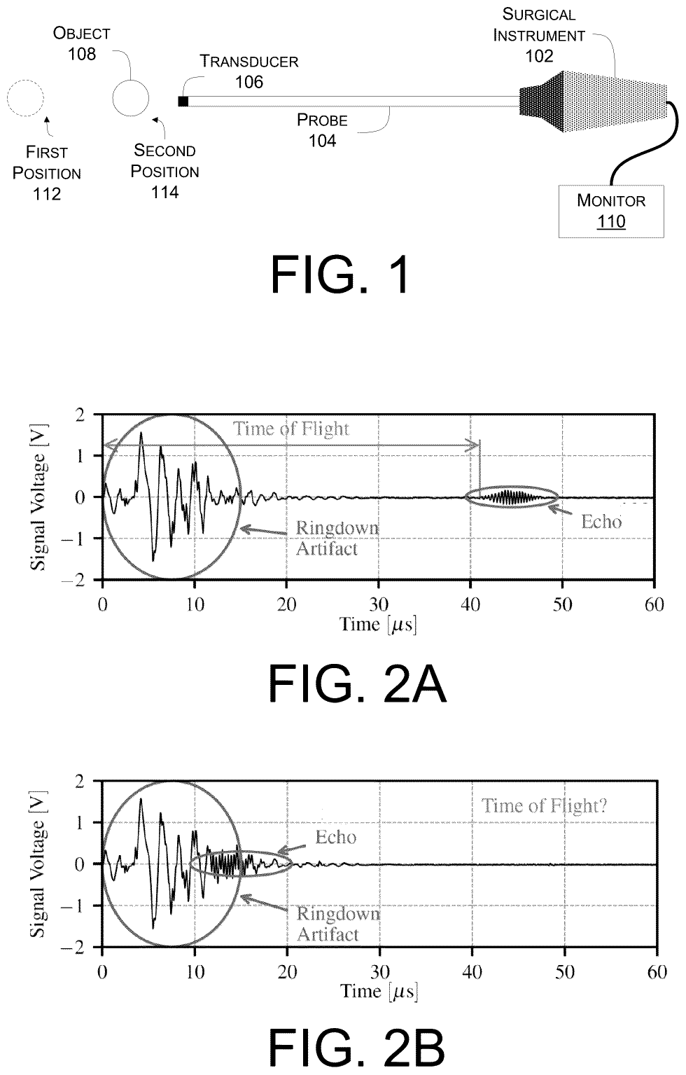

FIG. 1 illustrates an example surgical environment 100 for detecting tissue characteristics using a surgical instrument 102. The surgical instrument 102, in some examples, is a laparoscopic instrument, a probe, a laparoscopic grasper, a laparoscopic instrument, a robotic surgery tool, an endoscopic tool, an arthroscopic tool, a video-assisted thoracoscopic surgery (VATS) tool, or a catheter. For example, the surgical instrument 102 includes a probe 104 configured to be inserted into a body of a subject through a trocar, incision, port, or a combination thereof.

In various implementations, the probe 104 includes a transducer 106 configured to detect an object 108 in the body of the subject. The transducer 106, for instance, is an ultrasound transducer. In various cases, the transducer 106 outputs an ultrasound signal toward the object 108. The object 108 reflects at least a portion of the ultrasound signal. The transducer 106, in various cases, detects the reflected portion of the ultrasound signal. In various cases, the surgical instrument 102 and/or a monitor 110 communicatively coupled with the surgical instrument 102, is configured to detect a distance between the probe 104 and the object 108 and/or a characteristic of the object 108. The monitor 110, for instance, includes an output device (e.g., a display, a microphone, a haptic feedback device, or the like) configured to output, to a user, a feedback signal indicative of the distance between the probe 104 and the object 108 and/or the characteristic of the object 108.

In various cases, the transducer 106 outputs an incident ultrasound signal in the form of a pulse toward the object 108. The transducer 106 may include a piezoelectric crystal electrically coupled to electrodes. In various cases, a driving circuit outputs an electrical signal to the electrodes during a first time period, which is finite. In various cases, the piezoelectric crystal vibrates in response to the electrical signal, which induces pressure waves in the surrounding environment. These induced pressure waves represent the incident ultrasound signal. Because the driving circuit activates the piezoelectric crystal for a finite time period, the incident ultrasound signal is transmitted as a pulse. The incident ultrasound signal is transmitted through the surrounding environment and is reflected back towards the transducer 106 by the object 108. In some examples, the interaction between the ultrasound signal and the object 108 changes characteristics of the ultrasound signal. For instance, physical characteristics of the object 108 may alter characteristics of the ultrasound signal as it is reflected from the object 108. That is, the echo (also referred to as the “reflection”) of the ultrasound signal may have different characteristics than the as-transmitted ultrasound signal.

After outputting the ultrasounds signal as a pulse, the transducer 106 may transition to a “listening” phase. When the echo of the ultrasound signal is received by the piezoelectric crystal of the transducer 106, the piezoelectric crystal converts the received pressure waves into an electrical signal that can be analyzed by the surgical instrument 102 and/or the monitor 110. For example, a time interval between the transmission of the incident ultrasound signal by the transducer 106 and the reception of the echo by the transducer 106 is proportional to the distance between the transducer 106 and the object 108 from which the ultrasound signal is reflected. This time interval, for instance, can be referred to as a “time-of-flight” (or “time of flight” or TOF) of the ultrasound signal. In another example, a frequency shift (referred to as a “Doppler shift”) between the incident ultrasound signal and the echo may be proportional to a velocity of the object 108 in a dimension parallel to the direction of the ultrasound signal. Thus, in some cases, the echo can be analyzed in order to identify the position and/or velocity of the object 108 in space.

However, in many cases, a ringdown artifact corrupts the detection of the echo by the transducer 106. In various cases, the ringdown artifact is caused by residual vibration of the piezoelectric crystal after the electrical signal stimulating the piezoelectric crystal is ceased. For instance, the piezoelectric crystal continues to vibrate after a driving circuit connected to the electrodes ceases to supply electrical energy to the electrodes. This prolonged ringing by the piezoelectric crystal induces an electrical signal in the electrodes. The electrical signal caused by the ringing, for instance, causes the ringdown artifact.

FIGS. 2A and 2B provide examples of detected signal voltages across the piezoelectric crystal of the transducer 106 of FIG. 1 with respect to time. FIG. 2A, for instance, illustrates the detected voltage when the object 108 is located at a first position 112, which is relatively far away from the transducer 106. FIG. 2A, for instance, illustrates that a ringdown artifact produces an oscillation in the voltage within about the first 15 microseconds (μs) of the listening phase of the transducer. However, because the object 108 is positioned at a sufficient distance from the transducer 106, the echo from the object 108 is received after the ringdown artifact is substantially reduced in the detected voltage. Accordingly, the ringdown artifact does not impede an estimate of the time-of-flight of the ultrasound signal when the object 108 is located at the first position 112.

FIG. 2B, in another example, illustrates the detected voltage when the object 108 is located at a second position 114, which is relatively close to the transducer 106. That is, the object 108 is positioned within a ringdown range of the transducer 106. In some cases, the ringdown range is a range of positions within centimeters (e.g., within two centimeters (cm)) of the transducer 106. An echo from any object within the ringdown range may overlap a ringdown artifact of the transducer 106, for instance. Due to the close proximity of the object 108 in this case, the ringdown artifact overlaps the reception of the echo from the object 108. Accordingly, the ringdown artifact can impede accurate detection of the time-of-flight of the ultrasound signal when it is reflected by the object 108 within close proximity of the transducer 106. The ringdown artifact may further impede accurate detection of other characteristics (e.g., a Doppler shift) of the echo from the object 108, which can impede the detection of other characteristics of the object 108 that would otherwise be ascertainable from the echo. If the ringdown artifact is not suppressed, the transducer 106 is unable to detect the echo from the object 108 when the object 108 is within a distance range of the transducer 106.

Various implementations of the present disclosure address these and other problems by filtering the ringdown artifact from data representing the detected voltage across the transducer 106. Accordingly, the surgical instrument 102 and/or the monitor 110 are able to detect the position and other characteristics (e.g., velocity) of the object 108 even when the object 108 is located within close proximity of the transducer 106.

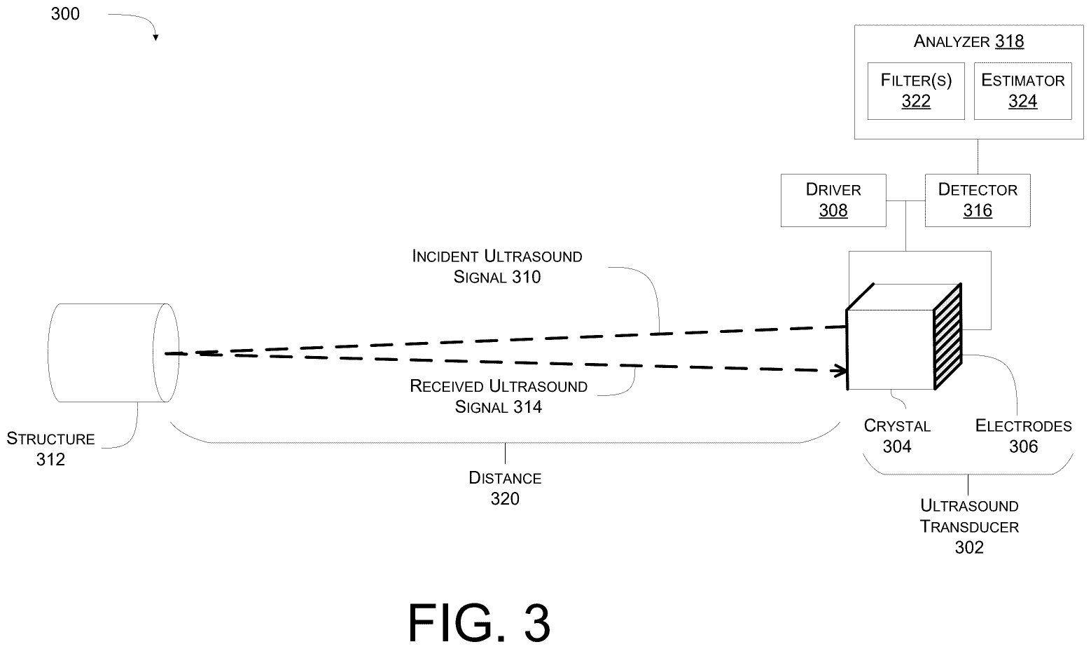

FIG. 3 illustrates a generic environment 300 for enhancing the detection range of an ultrasound transducer 302 by suppressing a ringdown artifact. The ultrasound transducer 302, for instance, can be the transducer 106 described above with reference to FIG. 1. The environment 300, in some cases, is within the body of a subject. For example, the ultrasound transducer 302 may be inserted into the body of the subject.

The ultrasound transducer 302 includes a crystal 304. The crystal 304, for instance, includes at least one piezoelectric material, such as Pb(Zr, Ti)O₃ (PZT), Pb(B′B″)O₃—PbTiO₃ (wherein B′=Mg²⁺, Zn²⁺, Ni²⁺, or the like; B″=Nb⁵⁺, Ta⁵⁺, W⁶⁺. . . ), quartz, LiNbO₃ (lithium niobate or LN), or any combination thereof.

In various cases, the crystal 304 is configured to exhibit a mechanical strain in response to the application of an electric field on the crystal 304. Electrodes 306 disposed on the crystal 304, for instance, are configured to apply a voltage across the crystal 304.

A driver 308 is a circuit configured to supply an electrical signal to the electrodes 306 that causes the electrodes 306 to output the voltage to the crystal 304. In various implementations, the driver 308 includes an integrated circuit, H-bridge, at least one amplifier, and filter (e.g., an RC filter configured to round edges of substantially square pulses generated by the rest of the circuit) configured to generate and output a periodic electrical signal to the electrodes 306. In some cases, when the driver 308 and the electrodes 306 vary the electric field periodically (e.g., wherein the voltage applied to the crystal 304 over time has a periodic waveform, such as a square wave or a sine wave), the crystal 304 vibrates at the same frequency as the periodic waveform. In some implementations, the crystal 304 vibrates at a resonant frequency. In various cases, the vibration of the crystal 304 generates pressure waves within the surrounding environment 100. These pressure waves, in various implementations, include sound waves. When the vibration of the crystal 304 and the pressure waves have a frequency that is greater than 20 kilohertz (kHz), the crystal 304 generates ultrasound in the environment 200.

Although not specifically illustrated in FIG. 1, the ultrasound transducer 302 may further include at least one impedance matching layer disposed on at least one of the electrodes 306, such that one of the electrodes 306 is disposed between the crystal 304 and the impedance matching layer. In various cases, the impedance matching layer includes at least one material with an acoustic impedance that is intermediate to the acoustic impedance of the crystal 304 and/or electrodes 306 and the surrounding environment 300. In some examples, the ultrasound transducer 302 further includes a fluid-tight housing that protects the rest of the ultrasound transducer 302 from damage and/or contamination from the environment 300.

In various implementations of the present disclosure, the ultrasound transducer 302 emits an incident ultrasound signal 310 in the environment 300. For example, the incident ultrasound signal 310 has a frequency in a range of 20 KHz to 4 megahertz (MHz), a range of 1 MHz to 4 MHz, or in a range of 2.5 MHz to 3.5 MHz. According to some cases, the incident ultrasound signal 310 is a pulse. For instance, the driver 308 is configured to apply the periodic electrical signal to the electrodes 306 in a pulse that has a duration in a range of 1 microsecond (μs) to 10 μs.

The incident ultrasound signal 310 is transmitted toward a structure 312 in the environment 300. The structure 312, for instance, includes a physiological structure within the body of the subject. Examples of physiological structures include organs, blood vessels, bones, tumors, embryonic tissue, fetal tissue, and the like. In some cases, the structure 312 includes an implant or other type of device within the body of the subject. In some examples, the structure 312 includes a fluid, such as blood, amniotic fluid, intracellular fluid, interstitial fluid, and the like.

At least a portion of the structure 312 interacts the incident ultrasound signal 310, thereby generating a received ultrasound signal 314 that is received by the ultrasound transducer 302. In some implementations, the received ultrasound signal 314 is the reflection of the incident ultrasound signal 310 from the structure 312. According to some cases, the incident ultrasound signal 310 and the received ultrasound signal 314 have different shapes, waveform shapes, frequencies, durations, phases, or other characteristics. In various cases, the different characteristics of the incident ultrasound signal 310 and the received ultrasound signal 314 are indicative of one or more characteristics of the structure 312.

In various examples, the ultrasound transducer 302 detects the received ultrasound signal 314. The received ultrasound signal 314, for instance, induces a mechanical strain in the crystal 304. In some cases, the mechanical strain causes the crystal 304 to vibrate. Due to the piezoelectric material(s) within the crystal 304, the mechanical strain caused by the received ultrasound signal 314 induces a voltage across the crystal 304 that is detected by the electrodes 306.

In various implementations, a detector 316 includes a circuit configured to detect the voltage across the crystal 304 after the driver 308 outputs the electrical signal that induces the incident ultrasound signal 310. For example, a period of time after the driver 308 is actively supplying the electrical signal to the electrodes 306 is referred to as a “listening phase.” In some cases, the detector 316 includes one or more analog-to-digital converters (ADCs) configured to convert samples of the voltage across the crystal 304 with respect to time into digital data.

An analyzer 318, in various examples, is configured to analyze the voltage across the crystal 304 during the listening phase. For instance, the analyzer 318 is configured to analyze the data representative of the voltage across the crystal 304 during the listening phase. In some cases, the analyzer 318 is executed by at least one computing device, at least one processor, as instructions stored in memory, or any combination thereof.

According to various cases, the ultrasound transducer 302 detects the received ultrasound signal 314 after the driver 308 outputs the electrical signal to the electrodes 306. However, in a brief time period after the driver 308 ceases actively outputting the electrical signal to the electrodes 306, the crystal 304 continues to vibrate or “ring.” In various implementations, the continued vibration of the crystal 304 causes the crystal 304 to output, to the electrodes an electrical signal that is unrepresentative of the received ultrasound signal 314. In various cases, the ringing of the crystal 304 generates a ringdown artifact in the detected voltage across the crystal 304 over time. In various cases, the ringdown artifact is also reflected in the data representative of the voltage across the crystal 304.

If a distance 320 between the ultrasound transducer 302 and the structure 312 is sufficiently long, then the ringdown artifact may end prior to the ultrasound transducer 302 detecting the received ultrasound signal 314. However, if the distance 320 is within a ringdown range of the ultrasound transducer 302, then the ringdown artifact may temporally overlap with the detection of the received ultrasound signal 314 by the ultrasound transducer 302. Although not specifically illustrated in FIG. 1, in some implementations, the distance 320 is representative of the path length of the incident ultrasound signal 310 and/or the path length of the received ultrasound signal 314. In some cases, the ringdown artifact may prevent the analyzer 318 from detecting or analyzing the received ultrasound signal 314.

In various implementations of the present disclosure, the impact of the ringdown artifact can be minimized by the analyzer 318 using at least one filter 322. In some cases, the filter(s) 322 is applied to the data representative of the voltage across the crystal 304 over time. For instance, the filter(s) 322 include at least one digital filter. In some cases the filter(s) 322 is multiplied or convolved with the data representative of the voltage across the crystal 304 over time. In some examples, the filter(s) 322 is multiplied or convolved with a frequency domain representation of the data representative of the voltage across the crystal 304 over time. Although the description of FIG. 1 primarily assumes the filter(s) 322 include digital filters, implementations are not so limited. For instance, the filter(s) 322 can be implemented as analog filters using various techniques known to those having ordinary skill in the art.

In some implementations, the filter(s) 322 include a bandpass filter. For instance, the bandpass filter may be configured to substantially maintain one or more frequency components of the received ultrasound signal 314 and may be configured to substantially suppress frequency components of the ringdown artifact. A pass band of the bandpass filter, in various cases, overlaps with a frequency of the received ultrasound signal 314 without being overlapping with a frequency of the ringdown artifact, in various cases. The bandpass filter, for instance, includes a Gaussian filter, a Butterworth filter, a Chebyshev filter, an elliptic filter, or any combination thereof.

In some cases, the filter(s) 322 include an adaptive filter, such as a least mean squares (LMS) filter, a recursive least squares (RLS) filter, a finite impulse response (FIR) filter, an adaptive linear combiner (ALC), a Wiener filter, or the like. In various cases, the adaptive filter includes weights that are dynamically adjusted during application to the data in order to minimize an error and/or loss. In particular cases, an LMS filter optimizes its coefficients by minimizing a least mean square of an error (e.g., calculated based on a difference between desired data and the data on which the filter has been applied). According to some cases, the filter(s) 322 include a spectrum suppression filter. In various implementations, the filter(s) 322 is generated using machine learning (ML). For instance, the filter(s) 322 include a data structure defined by various parameters that can be optimized to remove the ringdown artifact when applied to the data. The parameters, for instance, are optimized based on training data.

The training data can be obtained in various ways. In some cases, the training data includes previously recorded instances of ringdown artifacts generated and/or detected by the ultrasound transducer 302. In some examples, the training data includes previously recorded instances of ringdown artifacts generated by ultrasound transducers that are similar to the ultrasound transducer 302. For example, the similar ultrasound transducers may include crystals with the same resonant frequency as the crystal 304. In various cases, the recordings of ringdown artifacts in the training data are generated by applying similar electrical signals to the ultrasound transducer 302 (or similar ultrasound transducers) as the electrical signal generated by the driver 308.

Various techniques can be utilized to train the ML-based data structure. For instance, the parameters of the data structure can be optimized using autoregression, gradient descent, or the like. Training can be performed using supervised learning, unsupervised learning, or a combination thereof.

Various types of data structures can be utilized as an ML-based filter among the filter(s) 322. In various cases, the ML-based filter includes a neural network (e.g., a convolutional neural network (CNN), a recurrent neural network (RNN), or the like), a hidden Markov model (HMM), or the like. According to some cases, the ML-based filter includes one or more layers, wherein each layer generates output data by processing input data via applying at least one transformation to the input data. The transformation(s) of the layer are defined by the parameters, which are optimized based on training data, for instance. Layers can be arranged in series (e.g., the output data of a first layer is input data to a second layer) and/or in parallel (multiple layers receive and process the same input data). Each layer, in various cases, includes one or more neurons associated with the transformation(s). In some cases, the filter(s) 322 include a long short-term memory (LSTM) filter and/or a bidirectional RNN (BRNN), gated recurrent unit (GRU), an encoder-decoder, an Elman network, a Jordan network, a Hopfield network, an echo state network (ESN), or any combination thereof.

In various implementations, the analyzer 318 is configured to substantially suppress the ringdown artifact in the data representative of the voltage across the crystal 304 using the filter(s) 322. Further, the analyzer 318 may include an estimator 324 configured to detect the distance 320 and/or a characteristic of the structure 312 by analyzing the filtered data.

In particular examples, the estimator 324 estimates a time-of-flight between the ultrasound transducer 302 and the structure 312. For example, the time-of-flight may refer to a sum of the time that the incident ultrasound signal 310 takes to travel from the crystal 304 to the structure 312 and the time that the received ultrasound signal 314 takes to travel from the structure 312 to the crystal 304. In various examples, the speed of the incident ultrasound signal 310 may be equivalent to the speed of the received ultrasound signal 314. Accordingly, the distance between the crystal 304 of the ultrasound transducer 302 and the structure 312 can be estimated by determining half of a product of the time-of-flight and the speed of both the incident ultrasound signal 310 and the received ultrasound signal 314.

One or more techniques can be utilized to detect the time-of-flight based on the filtered data. In some examples, the estimator 324 performs magnitude thresholding, envelope peak detection, cross-correlation, or any combination thereof, to determine the time-of-flight based on the filtered data. In some cases, the estimator 324 is configured to transform the filtered data into the frequency domain (e.g., using a short-time Fourier transform) and to detect the time at which the received ultrasound signal 314 encounters the crystal 304 by analyzing the frequency domain representation of the filtered data. In various cases, the time-of-flight represents the time interval from the beginning of the listening phase to the time at which the received ultrasound signal 314 is received by the crystal 304 of the ultrasound transducer 302.

In various cases, the estimator 324 is configured to detect other features of the structure 312 based on the filtered data. For example, if the incident ultrasound signal 310 is reflected by a portion of the structure 312 that is moving (e.g., flowing blood in the structure 312) in a direction with a component that is parallel to the incident ultrasound signal 310, then a phase and/or frequency of the received ultrasound signal 314 may be shifted compared to the incident ultrasound signal 310 due to the Doppler effect. In various cases, a speed of the moving portion of the structure is proportional to a magnitude of the shift. Accordingly, in some cases, the estimator 324 may determine a speed of the portion of the structure 312 by comparing the filtered data to data representing the as-transmitted incident ultrasound signal 310 (e.g., data representing the voltage applied by the driver 308 to the electrodes 306 to generate the incident ultrasound signal 310).

In various cases, other characteristics of the structure 312 can be ascertained by the estimator 324 performing an analysis on the filtered data. Example characteristics include a density (e.g., a mass per volume), a compressibility, a rigidity, an elasticity, a stiffness, a viscosity, a strength, or an acoustic attenuation of the structure 312. In some implementations, a discrepancy between the acoustic attenuation of the structure 312 and the acoustic attenuation of the surrounding environment 300 impacts the amount of energy in the incident ultrasound signal 310 that is reflected as the received ultrasound signal 314. Accordingly, the amplitude of the voltage produced when the crystal 304 receives the received ultrasound signal 314 can be utilized to determine the acoustic attenuation of the structure 312. In some cases, the estimator 324 can determine if the structure 312 includes a material with a relatively low acoustic attenuation (e.g., soft tissue) or a material with a relatively high acoustic attenuation (e.g., bone) based on a similar analysis. In some cases, the overall shape of the envelope of the data representing the voltage corresponding to the absorption of the received ultrasound signal 314 by the crystal 304 indicates other physical properties (e.g., compressibility, size, shape, resonant frequencies, etc.) of the structure 312.

In some aspects, the size of the structure 312 can be determined. In some cases, a thickness of the structure 312 (e.g., in a direction at least partially parallel to the incident ultrasound signal 310 and/or the received ultrasound signal 314) can be determined by analyzing the filtered data. For example, in an industrial process, producing a sheet or web of material, it may be desirable to use ultrasound to continuously measure or monitor thickness of the produced material. Although not specifically illustrated in FIG. 3, in some implementations, the ultrasound transducer 302 is applied directly (e.g., physically coupled) to one surface of the structure 312, such that the incident ultrasound signal 310 is transmitted through the structure 312, and the received ultrasound signal 314 is reflected from another surface of the structure 312. The thickness, for instance, is defined along the transmission path of the ultrasound through the structure 312. In various cases, the travel time for the return of the received ultrasound signal 314, with knowledge of speed of sound in the material, gives a measure of the thickness of the structure 312. If the structure 312 has a relatively short thickness (e.g., the structure 312 is relatively thin), a ringdown artifact could obscure the detection of the received ultrasound signal 314. In some cases, the ringdown artifact can be avoided by inserting one or more spacers (e.g., materials configured to transmit the incident ultrasound signal 310 and the received ultrasound signal 314) between the ultrasound transducer 302 and the structure 312, in order to prevent the crystal 304 from detecting the received ultrasound signal 314 as the crystal 304 is ringing. However, the use of spacers has several disadvantages. In various cases, the incident ultrasound signal 310 and the received ultrasound signal 314 are attenuated by additional travel through the spacer(s). In addition, the spacer(s) would make a device including the ultrasound transducer 302 bulkier and more expensive. In various cases, by analyzing the filtered data for the thickness of the structure 312, the thickness of the structure 312 can be ascertained in a compact and relatively inexpensive device.

In some examples, the estimator 324 may include a classifier used to identify a characteristic of the structure 312 using the filtered data. In some implementations, the classifier includes a ML-based classifier, such as a decision tree, a naïve Bayes classifier, a k-nearest neighbor model, a support vector machine, an ANN, or a transformer. For example, parameters of the ML-based classifier have been previously optimized during training to enable the classifier to determine the characteristic. In various cases, the ML-based classifier was trained using a supervised learning technique and based on training data indicating filtered data and characteristics obtained for other objects. For instance, the training data may include filtered data representing echoes detected from objects having various compressibilities, and the training data may further include separately measured compressibilities of the objects. In various cases, the filtered data representing the echoes is fed into the ML-based classifier, and the outputs of the ML-based classifier are compared to the measured compressibilities. During training, various parameters of the ML-based classifier are optimized in order to minimize a loss between the outputs of the ML-based classifier and the measured compressibilties.

One or more types of input data can be provided to the ML-based classifier. In some examples, the ML-based classifier of the estimator 324 receives an entire set of the filtered data in the time domain and/or the frequency domain. In some aspects, the ML-based classifier of the estimator 324 receives an envelope of the filtered data. In some examples, the ML-based classifier receives a portion of the filtered data in the time domain and/or frequency domain. According to some cases, the ML-based classifier also receives an indication of the electrical signal output by the driver 308 to generate the incident ultrasound signal 310. The ML-based classifier, for instance, outputs an indication of the characteristic of the structure 312 by performing various functions on the input data.

According to some implementations, the estimator 324 identifies the location and/or characteristic(s) of the structure 312 based on the filtered data and additional information. According to various cases, the additional information determined by a sensor disposed in the environment 300. In some cases, a temperature of the environment 300 may impact the filtered data. For example, the speed of the incident ultrasound signal 210 and the received ultrasound signal 314 in the environment 300 can be changed based on the temperature of the environment 300. In some examples, a thermometer (not illustrated) is disposed in the environment 300. A temperature detected by the thermometer, for instance, can be further utilized to enhance the analysis of the filtered data. In various examples, the additional data is input into the ML-based classifier in order to enhance the determination of the characteristic of the structure 312.

In some examples, the ultrasound transducer 302, the driver 308, the detector 316, and the analyzer 318 are incorporated into one or more devices. For instance, a standalone device (e.g., a standalone surgical instrument, surgical robot, scope, or the like) may include the ultrasound transducer, the driver 308, the detector 316, and the analyzer 318. Alternatively, these components may be distributed among multiple devices (e.g., a surgical instrument communicatively coupled to a remote server or monitor). In some cases, the device(s) further include an output device, such as a display (e.g., a screen, an augmented reality (AR) display, a virtual reality (VR) display, or the like), a microphone, or a haptic feedback device (e.g., a device configured to be held by a user and which selectively vibrates). The output device, in various implementations, may output an indication of the distance 320 and/or the characteristic(s) of the structure 312. In some cases, the indication is output to a user substantially in real-time, with limited (e.g., one or more microseconds, milliseconds, or the like) latency after the received ultrasound signal 314 is received by the crystal 304.

Although FIG. 3 is described with reference to an ultrasound transducer 302 with a single crystal 304, implementations are not so limited. In some cases, the ultrasound transducer 302 can include an array of crystals including the crystal 304. Using various techniques described herein, the ringdown artifact of a crystal in the array can be suppressed to enable the detection of reflections of ultrasound signals transmitted by other crystals in the array, as well as situations in which a single crystal serves as the transmitter and receiver. In some cases, various ringdown artifact suppression techniques described herein can be utilized to enhance ultrasound imaging of the environment 300, such that images of even the ringdown range of the ultrasound transducer 302 can be obtained. In some examples, techniques described herein can be utilized to remove ringdown artifact for continuous wave (CW) ultrasound transducers.

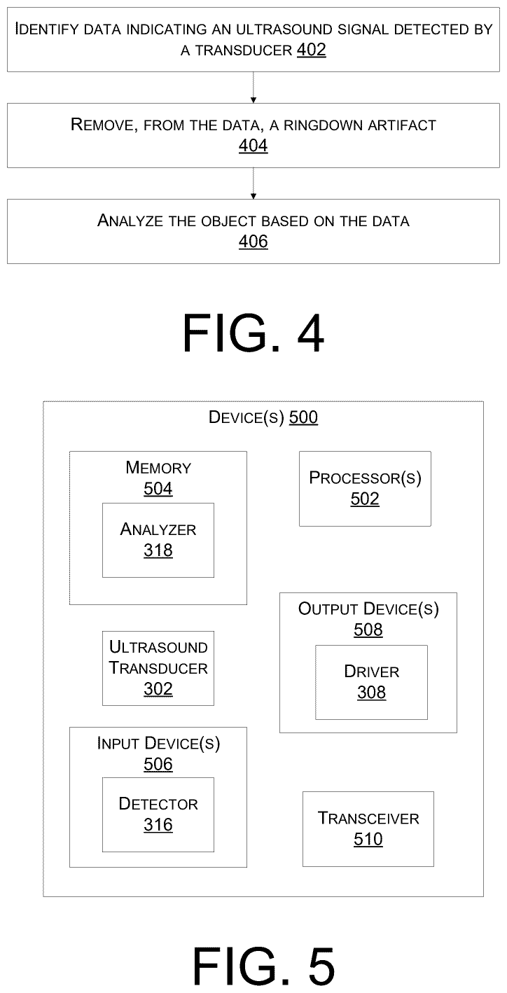

FIG. 4 illustrates an example process 400 for enhancing ultrasound detection of an object using ringdown artifact suppression. The process 400 is performed by an entity, which may include at least one processor, at least one computing device, at least one medical device, the surgical instrument 102, the monitor 110, the ultrasound transducer 302, the driver 308, the detector 316, the analyzer 318, or any combination thereof.

At 402, the entity identifies data indicating an ultrasound signal detected by a transducer. For example, the data represents samples of a voltage across a piezoelectric crystal of the transducer over time when the ultrasound signal is absorbed by the piezoelectric crystal. In some cases, the entity itself includes the transducer and generates the data (e.g., by converting the voltage samples into data using an ADC). Optionally, the transducer transmits an incident ultrasound signal that is reflected from an object. The reflection of the ultrasound signal, for instance, is the ultrasound signal detected by the transducer. In various cases, the incident ultrasound signal has a frequency in a range of 2.5 MHz to 3.5 MHz.

In various cases, the object is in close proximity with the transducer. For example, the object is 2 cm or less from the transducer. In some cases, the object is in a range of 0.1 cm to 2 cm, a range of 0.5 cm to 1.8 cm, or a range of 0.8 cm to 1.5 cm of the transducer. The object may include a physiological structure, such as a soft tissue, a bone, a foreign body, or a tumor. In some aspects, the object includes a medical device (e.g., an implantable device) disposed within a subject.

At 404, the entity removes, from the data, a ringdown artifact. In various implementations, the entity is configured to apply a digital filter and/or an artifact suppression technique. In various implementations, the entity applies at least one of a bandpass filter, an adaptive LMS filter, an SPS, a GRU, an LSTM, or an RNN to the data. According to some cases, the entity applies at least one ML-based filter to the data. The ML-based filter may be pre-trained. For example, the training data used to train the ML-based filter may be obtained by detecting previous ultrasound signals (e.g., in a controlled environment).

At 406, the entity analyzes the object based on the data. In some cases, the entity determines a time-of-flight between the transducer and the object. For instance, the entity uses a cross-correlation or minimum threshold technique to determine the time-of-flight. In various cases, analyzing the object may include determining a velocity of the object, determining a distance between the transducer and the object, determining a size of the object, determining a thickness of the object, determining a mechanical characteristic of the object, or identifying the object. In some aspects, the entity uses an ML-based classifier to identify the object. For instance, the ML-based classifier is pre-trained using training data indicative of ultrasound signals reflected from various objects. At least one of the various objects may have similar characteristics to the object being identified after training, for instance. In some cases, the entity further analyzes the object based on additional information (e.g., temperature of the transducer and/or object), such as a signal detected from an additional sensor (e.g., a thermometer).

In various cases, the entity outputs a feedback signal indicative of the object. For instance, the feedback signal may indicate a characteristic, distance, or identity of the object determined based on the data. In some examples, the feedback signal includes a visual signal, a tactile signal, or an audio signal. In some cases, the entity further transmits, to an external device (e.g., an external monitor, computing device, mobile device, or the like) a communication signal indicative of the object. For instance, the communication signal is encoded with data that indicates the characteristic, distance, or identity of the object.

FIG. 5 illustrates at least one device 500 configured to execute various functions described herein. For example, the device(s) 500 may include the surgical instrument 102 and/or the monitor 110 described above with reference to FIG. 1. The device(s) 500, in various implementations, includes the ultrasound transceiver 302 described above with reference to FIG. 3.

The device(s) 500 include at least one processor 502 configured to execute instructions stored in memory 504. In various implementations, the processor(s) 502 include a central processing unit (CPU), a graphics processing unit (GPU), or both CPU and GPU, or other processing unit or component known in the art. The memory 504, for instance, includes removable storage and/or non-removable storage. The memory 504, for instance, includes Random Access Memory (RAM), Read-Only Memory (ROM), Electrically Erasable Programmable Read-Only Memory (EEPROM), flash memory, or other memory technology, Compact Disk Read-Only Memory (CD-ROM), Digital Versatile Discs (DVDs), Content-Addressable Memory (CAM), or other optical storage, magnetic cassettes, magnetic tape, magnetic disk storage or other magnetic storage devices, or any other medium which can be used to store the desired information and which can be accessed by the device(s) 500. Any such tangible computer-readable media can be part of the device(s) 500.

The device(s) 500 further include at least one input device 506 and/or at least one output device 508. For instance, the input device 506 includes one or more sensors, a keypad, a cursor control, a touch-sensitive display, voice input devices, or any combination thereof. In some cases, the output device(s) 508 include at least one display, a speaker, a printer, an actuator, at least one light source, or any combination thereof. In various cases, the input device(s) 506 include one or more ADCs and/or the output device(s) 508 include one or more digital-to-analog converters (DACs). In various implementations, the input device(s) 506 include the detector 316 and the output device(s) 508 include the driver 308.

In various implementations, the device(s) 500 include a transceiver 510 configured to transmit and/or receive communication signals with one or more external devices (not illustrated). For example, the transceiver 510 can include a network interface card (NIC), a network adapter, a Local Area Network (LAN) adapter, or a physical, virtual, or logical address to connect to various network components, for example. To increase throughput when exchanging wireless data, the transceiver 510 can utilize multiple-input/multiple-output (MIMO) technology. The transceiver 510 can comprise any sort of wireless transceivers capable of engaging in wireless, radio frequency (RF) communication. The transceiver 510 can also include other wireless modems, such as a modem for engaging in WI-FI™, WiMAX, BLUETOOTH™, infrared communication, and the like. The transceiver 510 may include transmitter(s), receiver(s), or both.

In various implementations of the present disclosure, the processor(s) 502 is configured to execute the analyzer 318 stored in the memory 504. Upon executing the analyzer 518, for instance, the processor(s) 502 may be configured to suppress and/or remove, from data generated by the detector 316 in the input device(s) 506, a ringdown artifact. For instance, the analyzer 318 includes at least one filter stored in the memory 504 that is applied to the data by the processor(s) 502. In response to suppressing and/or removing the ringdown artifact, the processor(s) 502 executing the analyzer 318 may determine a distance between the ultrasound transducer 302 and an object and/or another characteristic of the object.

### EXPERIMENTAL EXAMPLE

MIS can benefit from having miniaturized sensors, such as ultrasound sensors, on surgical graspers to provide additional information to the surgeons. A problem with ultrasound sensors is that a ringing artifact arises from decaying oscillation of the piezo element, and at short travel distances, the ringing artifact blends with the acoustic echo. Without a method to remove the ringing artifact from the blended signal, it is impossible to measure one of the main characteristics of an ultrasound waveform—Time of Flight (TOF).

This Experimental Example provides example results of testing various techniques for removing and/or minimizing ringing artifact. In this Experimental Example, a 6 mm ultrasound transducer was added to a surgical grasper, intended to measure acoustic properties of the tissue. However, the ultrasound sensor has a ringdown artifact arising from the decaying oscillation of its piezo element, and at short travel distances, the artifact blends with the acoustic echo. Without removal, the ringdown artifact prevents estimation of the time-of-flight. In this Experimental Example, six filtering methods to clear the artifact from the ultrasound waveform were compared: bandpass filter, Adaptive Least Mean Squares (LMS) filter, Spectrum Suppression (SPS), Recurrent Neural Network (RNN), Long Short-Term Memory (LSTM) and Gated Recurrent Unit (GRU). Following each filtering method, four time-of-flight extraction methods were compared: magnitude threshold, envelope peak detection, cross-correlation, and Short-time Fourier transform (STFT). In these tests, the RNN with cross-correlation method pair was shown to be optimal for this task, performing with the root mean square error of 3.6%. Specific examples of this Experimental Example are provided in Sosnovskaya et al., 2023 IEEE Sensors Applications Symposium (SAS), Ottawa, ON, Canada, 2023, pp. 1-6.

### I. INTRODUCTION

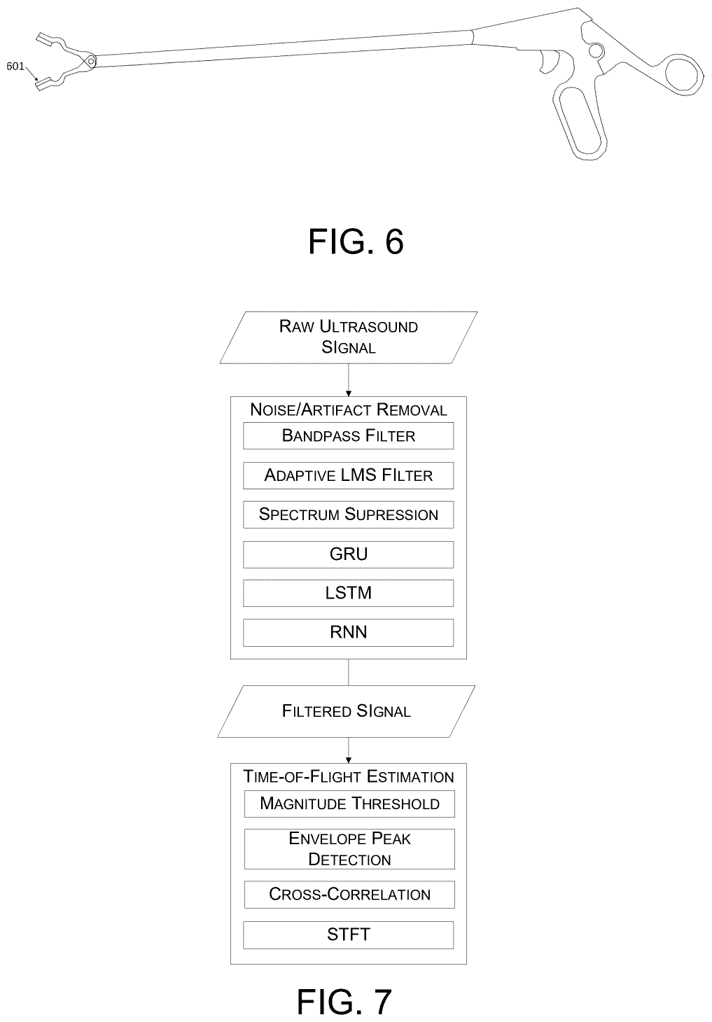

FIG. 6 illustrates an example of a customized surgical Babcock grasper equipped with an ultrasound transducer for testing in the Experimental Example. In this Experimental Example, a miniaturized, one-dimensional ultrasound transducer (i.e., a Steminc SMD063T07R111) was added to the tip of a surgical Babcock grasper. The ultrasound transducer was used to mechanically interface with modeled tissue and to measure its acoustic properties. A system-on-chip (i.e., a Texas Instruments TDC1000) was used to drive the ultrasound transducer; an external (i.e., 6 MHZ) clock to drive the transducer at a frequency suitable for imaging (e.g., 3 MHz) was used. Data was acquired using an oscilloscope (i.e., a Siglent SDS 1104X-E with a sampling rate of 500 MHZ). Ringdown artifacts in the data were successfully suppressed in order to achieve accurate time-of-flight estimation.

In this Experimental Example, the ultrasound transducer was used in bidirectional A-mode, which switches between transmitter and receiver functionalities. Operation in bidirectional mode saves space on the surgical grasper's jaws by using only one transducer instead of two. However, it is also a drawback: the system records exponentially decaying “ringing” (the “ringdown artifact”) of the transducer after excitation. When a surgical grasper's jaws operate on a tissue, the distance between them can be under 1 cm.

Short-distance ultrasound, in which the echo can blend with the ringdown, is applicable in many fields. These include the estimation of the degenerative loss of skeletal muscles (Qu et al., Proc. SPIE, Medical Imaging 2017: Ultrasonic Imaging and Tomography, vol. 10139, pp. 313-20, 2017), obstacle avoidance and mapping ultrasound sensors for underwater robots (Wirtz et al., 67^{th} international Astronautical Congress (IAC), 2016), robot position measurement (J. F. Figueroa, Ph.D. dissertation, Dept. Mech. Eng., Penn. St. Univ., 1988), thickness and defect detection applications in non-destructive testing (Wagle et al., Exp. Mech., vol. 51, no. 9, pp. 1559-1564, 2011), blood coagulation analysis (Voleisis et al., Ultrasonics, vol. 40, no. 1-8, pp. 101-107, 2002), and other ultrasound applications. All of these applications fundamentally suffer from being unable to accurately measure short TOFs due to the presence of ringdown artifacts.

This Experimental Example focuses on comparing and quantifying the performance of multiple ultrasound ringdown artifact removal techniques, and the subsequent time-of-flight estimation methods. The goal of this example is to evaluate and compare several signal processing and AI-based methods for clearing the corrupted ultrasound waveforms to accurately estimate the TOF. FIG. 7 illustrates various noise/artifact removal and time-of-flight estimation techniques tested in the Experimental Example.

### II. RELATED WORK

### A. Noise Removal Methods

Algorithms for noise removal can be divided into traditional filters and AI-based methods. Limited published literature exists on ringdown artifact removal from A-mode ultrasound signals, and even less on AI-based noise removal algorithms for ultrasound waveforms. One example is the gastrointestinal capsule with a 1D ultrasound for microanatomical diagnostics (Norton et al., Sci. Robot., vol. 4, no. 31, Jun. 2019). The capsule uses an additional ringdown-compensating filter by subtracting a moving average of the ringdown portion of the signal. This approach can help at longer TOFs, but not at short ones: at short TOFs, the ringdown blends with the echo, and the moving average starts including the echo as well.

Due to the similarity of ultrasound and conventional audio signals, some traditional audio algorithms successfully used in speech, natural language processing, translation and music could potentially work for ultrasound signals as well. A basic method, widely used in headphones and smartphones, consists of a device recording the noise separately, usually with a different microphone, and subtracting the noise from the obtained signal (Boll, IEEE T. Acoust. Speech., vol. 27, no. 2, pp. 113-20, 1979). An ultrasound's ringdown can be recorded separately, which makes this method feasible for ringdown artifact removal.

Frequency-selective (e.g., lowpass, highpass and bandpass) filters are frequently used for denoising signals, but they rely on the separation of the signal and the noise in the frequency domain. They may indeed be separate in certain ultrasound configurations, and therefore such filters are worth exploring.

Other traditional algorithms for overcoming the noise removal problem include adaptive filters (Widrow et al., Proc. IEEE, vol. 63, no. 12, pp. 1692-16, 1975). Examples of adaptive filters are Least Mean Squares (LMS), Wiener and Kalman filters. Such filters self-optimize based on a desired target signal, similar to deep learning methods. Obtaining such target signals may be nontrivial for ultrasound signals, but such filters do not have the other fundamental limitations listed above.

AI-based approaches for noise removal in 1D waveforms (sound, music, speech, and the like) include deep learning methods that work with time series data and imaging representation of time series data. RNNs work successfully with time series for speech denoising (Abdulbaqi et al., in Proc. ICASSP, Barcelona, Spain, pp. 6659-6663, 2020; Wu et al., in Proc. INTERSPEECH, pp. 3379-83, 2017). Time series signals can be transformed to 2D time-frequency representation based on Short-time Fourier Transform (STFT) and treated as images. Deep Convolutional Neural Networks (DCNN) are used with the images to clear the signal from the noise. Several studies have used DCNN to clear speech signals from corrupted noise (Kumar et al., in Proc. INTERSPEECH, pp. 3738-42, 2016), as well as ultrasound signal from the noise in the automotive industry (Mohamed et al., in Proc. VTC2019-Spring, pp. 1-6, 2019).

### B. Time of Flight Estimation Methods

TOF is the time required for a sound wave to travel through a medium. TOF depends on the distance traveled, and the medium's properties, including density, compressibility and rigidity, which are temperature-dependent. The velocity of sound is the distance traveled divided by TOF (P. Fish, Physics and Instrumentation of Diagnostic Medical Ultrasound. New York, NY, USA: John Wiley & Sons, 1990):

$$c = \frac{2 \times d}{t} \qquad ( 1 )$$

where c is the velocity of sound, d is distance traveled (doubled to account for the echo's two-way travel) and t is the TOF.

There exists a substantial body of published work on the estimation of TOF from a raw or processed signal. Some of these methods include:
Magnitude Threshold Method, used widely in hardware setups for automatic detection of TOF. When the amplitude of the received signal reaches a preset threshold, it records the time. The threshold should bypass the maximum noise level expected. This method is sensitive to noise fluctuations and signal decay with distance traveled, but is the most straightforward to implement (J. F. Figueroa, Ph.D. dissertation, Dept. Mech. Eng., Penn. St. Univ., 1988; Svilainis, in Proc. NDT, pp. 1-12, 2013).
Envelope Peak Detection includes approximating the echo signal as an analytical signal with complex components, from which a Hilbert envelope is computed. Then, peaks are found in the signal's envelope. The TOF is then detected either by locating the peak with the largest amplitude, or the first peak above a preset threshold. Such envelope signal is more robust to fluctuations of the echo's peaks, as well as a variable white noise level (J. F. Figueroa, Ph.D. dissertation, Dept. Mech. Eng., Penn. St. Univ., 1988; Gammell, Ultrasonics, vol. 19, no. 2, pp. 73-76, 1981).
In the Cross-correlation Method, in its simplest form, includes cross-correlating the transmitted and received signals, and estimating the TOF as the maximum in the correlation function. The method is generally robust, but can be sensitive to the quality of the transmitted signal (J. F. Figueroa, Ph.D. dissertation, Dept. Mech. Eng., Penn. St. Univ., 1988; Jia et al., Appl. Sci., vol. 9, no. 7, Art. no. 1483, 2019). Numerous modifications of this method exist, some are listed below.
Short-time Fourier Transform Method (STFT) for TOF estimation uses the time-frequency representation of ultrasound signals. STFT is obtained with a sliding window over the time domain signal, and taking the Fourier transform of the window. Then, windows with the frequency of interest are considered for the estimation of TOF (Lu et al., Ultrasonics, vol. 103, Art. no. 106104, 2020).

More advanced TOF estimation methods than those listed above exist in literature, including the use of Kalman filters to parameterize the signal (Angrisani et al., IEEE T. Instrum. Meas., vol. 55, no. 4, pp. 1077-1084, 2006), using a wavelet network to approximate the cross-correlation function (Grimaldi, IEEE T. Instrum. Meas., vol. 55, no. 1, pp. 5-13, 2006), refining the cross-correlation method by quantifying the phase shift of the received signal (Gueuning et al., IEEE T. Instrum. Meas., vol. 46, no. 6, pp. 1236-1240 Dec. 1997), and a Hilbert envelope-based method that identifies the time of zero envelope between a pair of pulse trains (Cai et al., IEEE T. Instrum. Meas., vol. 42, no. 6, pp. 990-994, Dec. 1993).

The methods discussed so far are generally designed for measuring long TOFs, well beyond those at which the ringdown artifact blends with the echo. Ringdown artifact removal techniques, as illustrated in FIG. 7 and discussed above, are therefore relevant for the success of any TOF estimation method. In this Experimental Example, the four simplest TOF estimation methods were paired with artifact removal methods. However, implementations of the present disclosure also apply to using other methods described herein.

### III. DATA COLLECTION AND PREPROCESSING

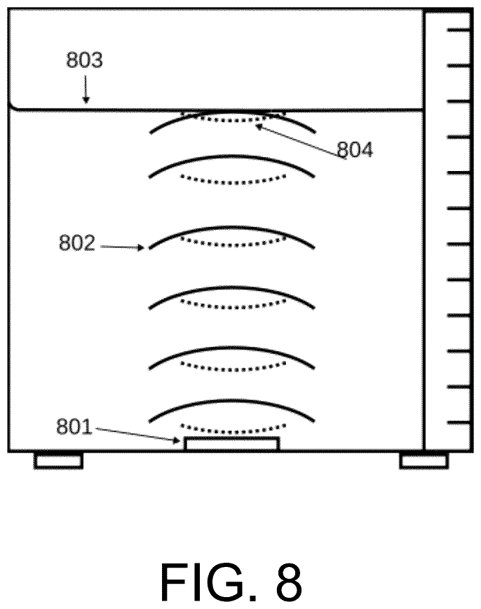

FIG. 8 illustrates another experimental setup that was utilized for data collection in the Experimental Example. This setup includes an acrylic container with an attached ruler to measure the level of liquid 803 in the container—representative of the one-way distance traveled. The same type of transducer 801 as the one used on the surgical grasper (see FIG. 6) was glued to the bottom of the acrylic container. Similarly, the same data acquisition setup was used: TI TDC1000 to drive the transducer at 3 MHZ, Siglent SDS 1104X-E oscilloscope to provide the external clock and acquire the signal. FIG. 8 illustrates the incident ultrasound signals 802 as well as the echoes 804 from the surface 803.

The data was recorded by the oscilloscope in csv format, combined and aligned in MATLAB. Then, the dataset was downsampled by a factor of 26 to a new sampling rate of 19.23 MHz, leading to 1279 samples per waveform.

In order to use the dataset for training in deep learning models, target signals were used for comparison and weight adjustment. When generating target signals, at distances of 2 cm and longer, the ringdown artifact and the detected echo were sufficiently far apart, which made it possible to isolate the echo by zeroing the ringdown time segment. The waveforms obtained at shorter distances were not used as target signals for the training dataset, due to the difficulty of extracting the echo from such signals. The training dataset contained 993 waveform pairs (with each pair including a raw signal, and a target signal with only the echo), with an approximately equal number of waveforms per distance.

A separate test dataset was collected at 9 distances from 0.5 cm to 4.0 cm, using liquid water as the medium. This testing dataset has 270 waveforms, 30 waveforms per distance. Both datasets were saved in mat format for further processing in Google Colaboratory with Python 3.6. Both the downsampled and original resolution datasets were published on IEEE DataPort (Sosnovskaya, IEEE Dataport, Online, 2022).

### IV. CRITERIA FOR METHOD COMPARISON

To quantitatively evaluate the results of the noise removal methods, two major criteria were used: Signal-to-Noise ratio (SNR) in frequency domain and TOF estimation. SNR in frequency domain shows how much the signal was cleared after applying denoising algorithms. SNR in frequency domain is calculated as:

$$SNR = 20 {log}_{10} \frac{{A}_{signal}}{{A}_{noise}} \qquad ( 2 )$$

with A_{signal} and Aₙₒᵢₛₑ being the signal and noise amplitudes, respectively. The signal's amplitude was taken in the frequency band [2.5 MHz, 3.5 MHz], where the oscillation frequency is located, and the noise was taken from the band [0, 2.5 MHz].

After denoising the ultrasound waveforms with the six noise/artifact removal techniques described in FIG. 7, TOF was estimated by four different methods: magnitude threshold, envelope peak detection, cross-correlation, and STFT. For the applications discussed in section I, the accuracy of the TOF is the main performance metric for the denoising and TOF estimation method pair. The reference TOF values were calculated using Eq. 1 with 1480 m/s as the velocity of sound in water at room temperature.

### V. DESCRIPTION OF NOISE REMOVAL TECHNIQUES

In this work, six noise removal techniques were tested: three traditional, and three deep learning ones for comparison. The traditional filtering techniques evaluated included the bandpass filter, the adaptive LMS filter, and the spectrum suppression method. The bandpass filter was designed with passband frequencies of 2.5 MHz to 3.5 MHz to bracket it over the 3 MHz resonant frequency. The adaptive LMS filter uses the target signal in order to optimize the coefficients for the filter. From preliminary attempts, the LMS filter order of 13 with a step size of 0.004 was selected for use on the signals-of-interest. Initial coefficients were based on the coefficients of the above bandpass filter with the Hamming window.

The Spectrum Suppression method used here was adapted from a paper by Boll (Boll, IEEE T. Acoust. Speech., vol. 27, no. 2, pp. 113-20, 1979). The method relies on a separate noise signal (i.e., the ringdown artifact measured from a free transducer) and the blended signal. The signals are transformed to the frequency domain via Fast Fourier Transform (FFT), and in frequency domain, the noise is subtracted from the blended signal. An inverse FFT is then used to obtain the denoised signal in time domain. Boll followed this sequence with a lowpass filter; in this Experimental Example, the above bandpass filter was used on the denoised signal instead.

For deep learning algorithms, RNN, LSTM, and GRU were chosen for comparison, because these algorithms work with time series data, and have previously been used for denoising speech signals. Between the three, RNN is the simplest and slowest method to train, LSTM have the most representational power, and are generally cheaper to train than standard RNNs, and GRU is a streamlined, cheaper-to-train version of LSTM. Chollet presents the theory of the three methods (Chollet, Deep Learning with Python. Shelter Island, NY, USA: Manning Publications, 2018).

In this example, the tanh activation function was used for all three deep learning networks, with the Adam optimizer with the learning rate of 5×10⁻⁴ and 330 training epochs. The Euclidean norm of the error between the input and target signals was used as the loss function. All trained networks had two layers with 512 and 256 units. All three networks used a bidirectional architecture.

### VI. RESULTS

### A. Noise Removal Comparison

The noise removal methods listed in Section V were tested on the 270-waveform testing data (see Section III for details).

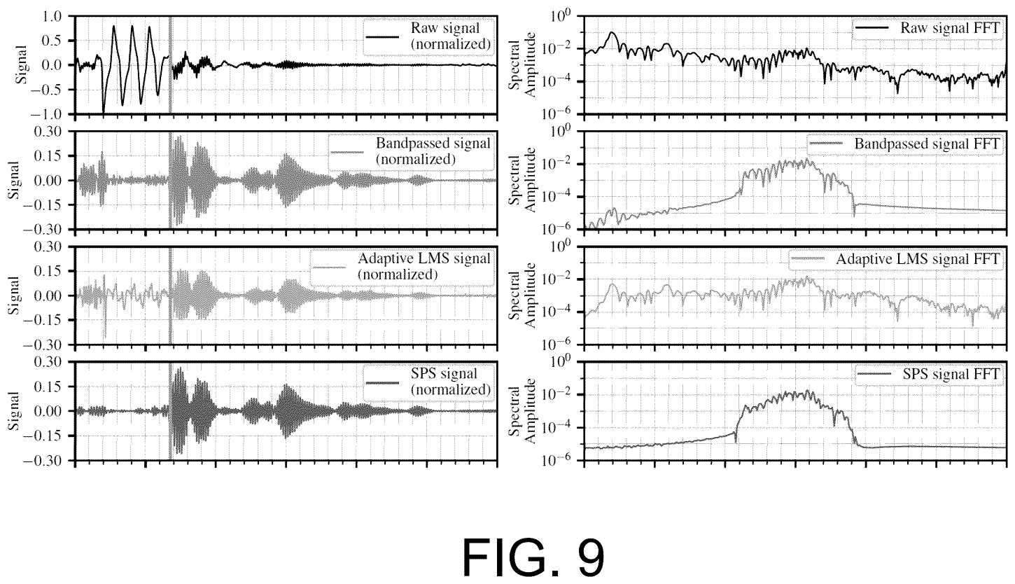

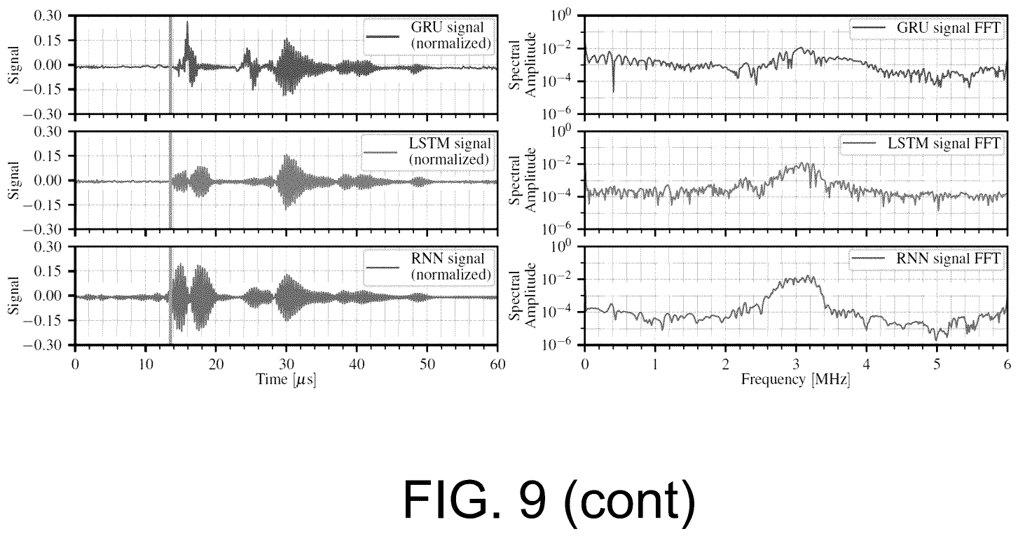

FIG. 9 illustrates results for various artifact removal techniques tested as part of the Experimental Example. Each method's results for artifact removal from raw ultrasound signals at 1 cm flight distance (here and below, this refers to the distance traveled until reflection and return to the transducer) are illustrated in FIG. 9. At this distance, the echo is capable of traveling back and forth multiple times, without decaying enough to become immeasurable; this multiecho phenomenon can be observed in all filtered signal plots, at approximately 13.5 μs, 27.0 μs and 40.5 μs. The training dataset did not have any data with multi-echo.

The upper left plot of FIG. 9 shows the raw signal, corrupted by the ringdown artifact; the echo's amplitude is smaller than the ringdown artifact's by about a factor of 3. At a shorter flight distance, this corruption would become more severe, thus making it even harder to extract the time of flight from the blended signal.

Because both the ringdown and the echo are physically generated by the oscillating transducer, part of the ringdown's frequency spectrum is similar to the echo's, and due to the short TOF, they blend with each other in time domain as well. For this reason, the bandpass filter still retains a lot of noise, particularly in the first 5 μs of the waveform. The adaptive LMS filter exhibits similar behavior. The spectrum suppression method, however, despite its reliance on the frequency domain, cleaned the signal much better, with only negligible noise remaining in the first 5 μs.

The performance of deep learning methods was more varied. As FIG. 9 shows, at a short distance, GRU significantly distorted the signal, although the ringdown artifact was removed completely. LSTM showed some distortion as well, although echoes remained visually recognizable. RNN performed the best among the three deep learning methods tested.

### B. Signal to Noise Ratio Comparison

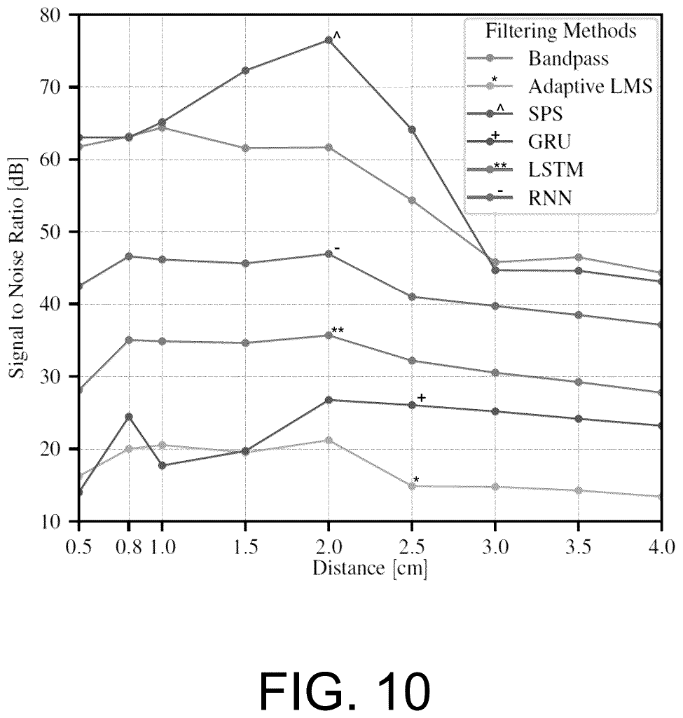

SNR was computed as discussed in Section IV. The results are presented on FIG. 10.

Among the techniques evaluated, the adaptive LMS filter exhibited the worst SNR characteristics, followed by GRU, which started to fail at distances shorter than 2 cm. The best SNR was observed for the bandpass filter, spectrum suppression, and RNN. The bandpass filter and spectrum suppression both filtered noise below 3 MHZ. The RNN learns to preserve this frequency, but still has some leftover noise at the lower frequency part of the spectrum (FIG. 10). It is also notable that the relative performance of all methods tested in this example stayed quite consistent across all travel distances, including those at which no appreciable blending of the echo and artifact occurred.

### C. Time of Flight Comparison

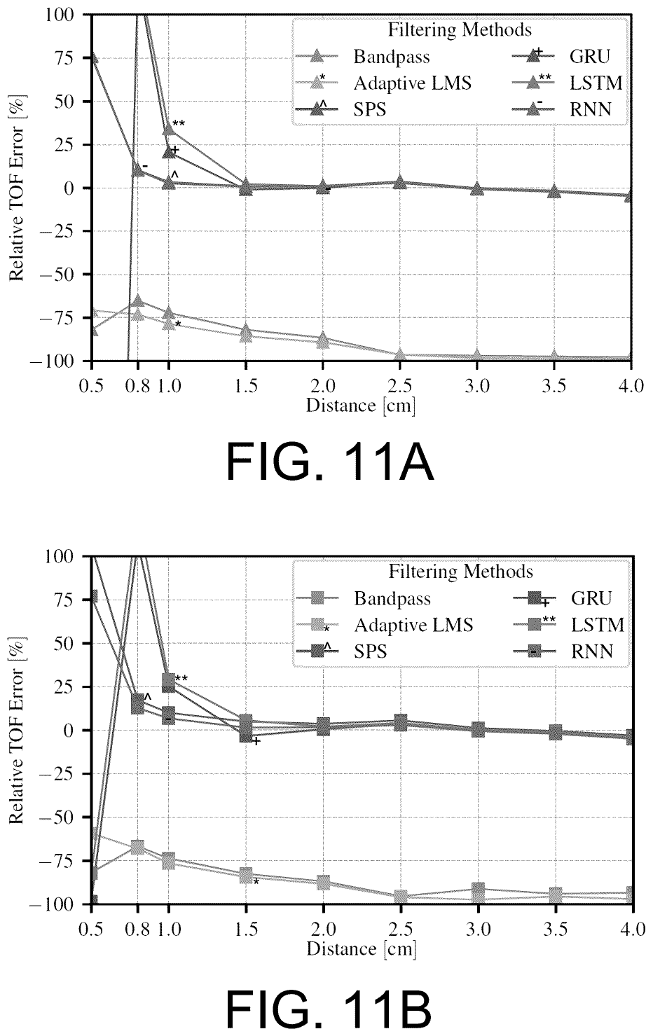

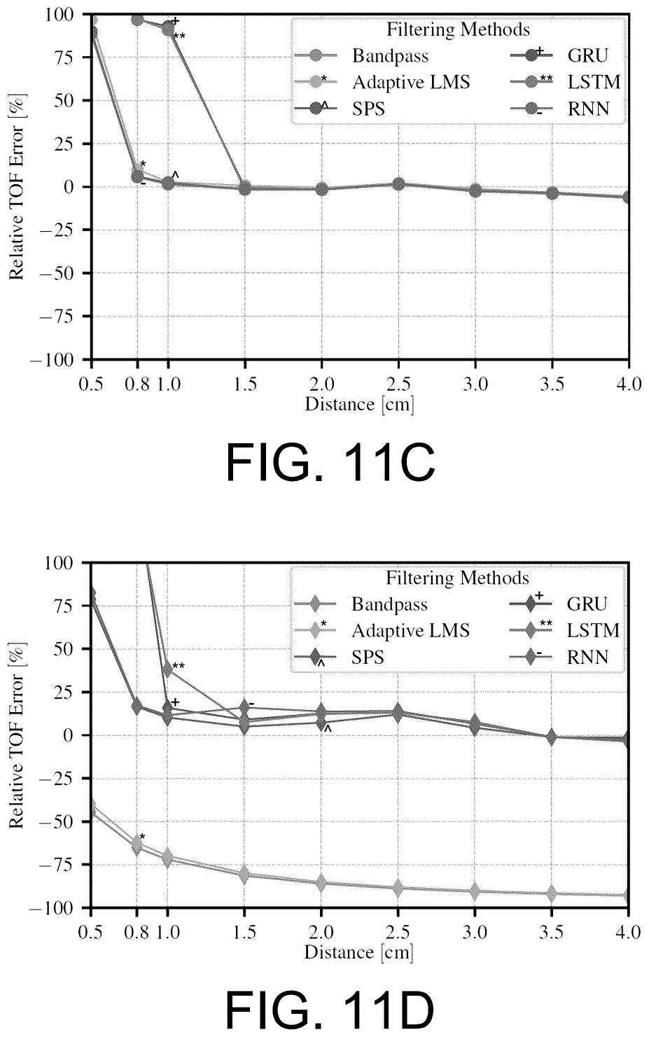

Following the denoising via the six methods discussed in Section V, TOF was estimated via the four methods discussed in Section II-B. Evaluating the true TOFs as described in section IV, mean relative errors in TOFs estimated by each method pair, for each distance measured. The results are presented FIGS. 11A to 11D.

As shown in FIGS. 11A to 11D, all pairs of methods that were evaluated (filtering method and TOF estimation method) fail at the distance of 0.5 cm, which indicates the system's limitation. It's observable that the deep learning denoising methods LSTM and GRU with the TOF estimation methods tested started to fail at distances lower than 1.5 cm. Distances lower than 2 cm were never shown during the training of deep learning denoising methods, and LSTM and GRU failed to generalize the solution for short-distance echo waveforms. It may be possible to use them with additional training data on short distances. On the other hand, standard RNN showed remarkably good results in spite of not having short-distance echo waveforms shown during the training. It should be understood that these limitations were representative of the specific methods tested, and that various techniques described herein could be adapted for identifying echo waveforms at shorter distances (e.g., distances of less than 1.5 cm, 1.0 cm, or 0.5 cm).

The tested bandpass and the adaptive LMS filters had relative errors of more than 75% paired with three of the four methods for TOF estimation at all distances. This is likely because as FIG. 9 illustrates, after these filters are applied, the remaining artifact is larger in amplitude than the echo, which causes threshold-based methods to fail. However, the bandpass and the adaptive LMS filters performed well with the cross-correlation TOF estimation method, because it does not depend on thresholding, but on signal correlation. The cross-correlation method is also more immune to white noise more than other methods tested.

Root Mean Square Error (RMSE) over all valid distances (i.e., 0.8 cm to 4.0 cm) was calculated for all 24 pairs of methods. 6 pairs demonstrated an RMSE of less than 5%:
1) Spectrum suppression with minimum threshold: RMSE of 4.31%.
2) RNN with minimum threshold: RMSE of 4.39%.
3) Bandpass with cross-correlation: RMSE of 3.62%.
4) Adaptive LMS with cross-correlation: RMSE of 4.40%.
5) Spectrum suppression with cross-correlation: RMSE of 3.70%.
6) RNN with cross-correlation: RMSE of 3.60%.

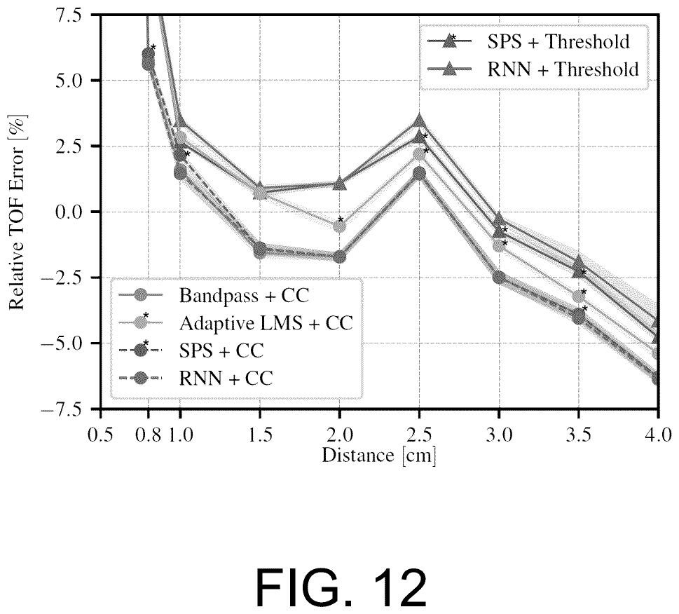

These methods' RMSEs for each distance, and their standard deviations over all samples taken at each distance are illustrated on FIG. 12. Based on their RMSEs, three pairs performed particularly well: bandpass with cross-correlation (CC), spectrum suppression with CC, and RNN with CC. RNN with CC has the lowest RMSE error of all the combinations of techniques tested.

### D. Statistical Analysis

Because the three best method pairs' RMSEs are so close to each other (within 0.1%), a statistical significance analysis of the difference between them is warranted. First, each method pair's relative error distribution (at each distance) was tested with the Kolmogorov-Smirnov normality test, which showed that the errors were not normally distributed. Because of this, the Wilcoxon signed rank statistical test was chosen to test if there is a statistical difference between the relative error distributions of the three best method pairs. Recall, that there are 30 waveforms for each analyzed distance.

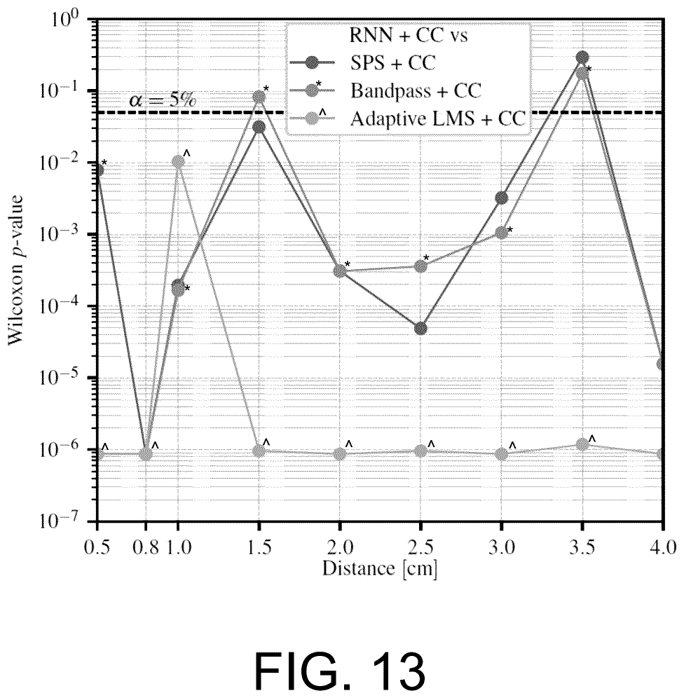

The results of the Wilcoxon test are presented on FIG. 13. Adaptive LMS with CC was also added to the statistical analysis, as an example of a clearly worse method, to frame the results. At most distances, the Wilcoxon signed rank test's p-value is clearly lower than, e.g., an α-value of 5%, which suggests that there is a statistically significant difference between the tested pairs of methods; again, these pairs are RNN with CC, SPS with CC, bandpass with CC and adaptive LMS with CC. At the distance of 3.5 cm, the test found no significant difference between RNN, SPS and bandpass filters.

### VII. DISCUSSION

The RNN denoising method, combined with the cross-correlation TOF estimation method, appear to be best tested technique suited for ringdown artifact removal, followed by TOF extraction. In literature, LSTM and GRU are typically considered superior methods to RNN due to the vanishing and exploding gradient problems (Chollet, Deep Learning with Python. Shelter Island, NY, USA: Manning Publications, 2018). At longer TOFs, with the echo and the ringdown artifact completely separated, GRU did indeed outperform all other methods. The poor performance of LSTM and GRU at shorter distances may be explained by the limitations of the training dataset, which, due to being generated by manually combining signals, did not have any examples of the multi-echo, and may have also lacked other features that the networks may have relied on.

RNN, on the other hand, showed optimal performance at both long and short distances, with results comparable to the traditional spectrum suppression and bandpass methods. It is conventional to compare neural network performance according to loss function values achieved on training sets, and their training time. Training time-wise, the results were indeed as expected, with RNN taking significantly longer than the other two, and GRU being the fastest. For this problem, however, the significant difference between the training and testing sets makes the loss function evaluated on the training set irrelevant; instead, average errors across the testing set, presented in the previous section, are more illustrative about the tested methods' relative performance.

### VIII. CONCLUSIONS

Six artifact removing methods were compared for the removal of ringdown artifacts from short distance A-mode ultrasound signals in liquid water: the bandpass filter, the adaptive LMS filter, spectrum suppression, RNN, LSTM and GRU. Their performance was evaluated based on SNR in frequency domain, and by estimating the TOFs from filtered signals. Four methods for extracting TOFs were compared: magnitude threshold, envelope peak detection, cross-correlation, and STFT.

The lowest RMSEs in TOFs among all 24 analyzed method pairs were observed from the RNN, spectrum suppression, and bandpass artifact removing methods, with each followed by the cross-correlation TOF estimation method. RNN with CC was the best performing method pair, and therefore it may be selected as the best method for future work for preprocessing the surgical grasper's data in further classification algorithms. The RMSE of this RNN with Cross-correlation method pair was 3.60% across distances of 0.8 cm and above.

Limitations of this example include a lack of temperature control: for calculating the true TOF. In this example, it was assumed that tests were performed in environments at a constant room temperature, but in practice it likely fluctuated. This could be addressed by adding a mounted thermistor to the second jaw of the surgical grasper, which could provide temperature as another input for deep learning algorithms.

Another potential limitation is the fact that the methods compared in this example were tested only on liquid water. It is anticipated that they will work on a wide range of fluids and tissues, as long as the distances and velocities of 3 MHz ultrasound remain similar, but further confirmations in other environments could be a subject of future inquiry.

### EXAMPLE CLAUSES

1. A laparoscopic instrument, including: a probe; an output device configured to provide a feedback signal; and a single ultrasound transducer disposed at an end of the probe and configured to: transmit an incident ultrasound signal; and detect a received ultrasound signal at least partially from an object within a range of about 0.5 centimeters to about two centimeters of the single ultrasound transducer; and at least one processor configured to: remove, from data indicative of the received ultrasound signal, a ringdown artifact; based on removing the ringdown artifact, determine, based on the data, a time-of-flight between the single ultrasound transducer and the object; and cause the output device to provide the feedback signal based at least in part on the time-of-flight.
2. The laparoscopic instrument of clause 1, wherein removing the ringdown artifact includes applying a recurrent neural network (RNN) to the data indicative of the received ultrasound signal; and wherein determining, based on the data, the time-of-flight between the single ultrasound transducer and the object includes estimating the time-of-flight by performing cross-correlation on the data.
3. The laparoscopic instrument of clause 1 or 2, wherein the output device includes at least one of a light, a display, a haptic feedback device, or a speaker; and wherein the feedback signal includes at least one of a visual signal, a tactile signal, or an audio signal.
4. At least one device, including: a single ultrasound transducer configured to: transmit an incident ultrasound signal; and detect a received ultrasound signal at least partially from a structure within a ringdown range the single ultrasound transducer; and at least one processor configured to: remove, from data indicative of the received ultrasound signal, a ringdown artifact; and based on removing the ringdown artifact, determine, by analyzing the data, a distance to the structure or a characteristic of the structure.
5. The at least one device of clause 4, wherein a frequency of the incident ultrasound signal is in a range of about 2.5 MHz to about 3.5 MHz.
6. The at least one device of clause 4 or 5, wherein the structure includes at least one of a soft tissue, a bone, a foreign body, or a tumor.
7. The at least one device of any of clauses 4 to 6, wherein the ringdown artifact includes a residual vibration of the single ultrasound transducer in response to transmitting the incident ultrasound signal.
8. The at least one device of any of clauses 4 to 7, wherein removing the ringdown artifact includes applying, to the data indicative of the received ultrasound signal, at least one of: a bandpass filter, an adaptive LMS filter, an SPS, a GRU, an LSTM, or an RNN.
9. The at least one device of any of clauses 4 to 8, wherein the characteristic of the structure includes at least one of: a velocity of blood in the structure; a velocity of a wall of the structure; a size of the structure; a thickness of the structure; a mechanical characteristic of the structure; or an identification of the structure.
10. The at least one device of clause 9, wherein analyzing the data includes: in response to removing the ringdown artifact from the data indicative of the received ultrasound signal, determining a frequency shift and/or a phase shift of the received ultrasound signal with respect to the incident ultrasound signal by analyzing the data; and determining a velocity of a fluid in the structure based on the frequency shift and/or the phase shift.
11. The at least one device of any of clauses 4 to 10, further including an output device configured to output an indication of the characteristic, wherein the output device includes at least one of a light source, a display, a speaker, or a haptic feedback device.
12. A method, including: transmitting, by a single ultrasound transducer, an incident ultrasound signal; detecting, at least partially from an object within a ringdown range of the single ultrasound transducer and by the single ultrasound transducer, a received ultrasound signal; removing, from data indicative of the received ultrasound signal, a ringdown artifact; and based on removing the ringdown artifact, analyzing the object based on the data.
13. The method of clause 12, wherein a frequency of the incident ultrasound signal is in a range of about 2.5 MHz to about 3.5 MHz.
14. The method of clause 12 or 13, wherein the object includes a soft tissue, a bone, a foreign body, or a tumor.
15. The method of any of clauses 12 to 14, wherein the ringdown artifact includes a residual vibration of the single ultrasound transducer in response to transmitting the incident ultrasound signal.
16. The method of any of clauses 12 to 15, wherein removing the ringdown artifact includes applying, to the data indicative of the received ultrasound signal, at least one of: a bandpass filter, an adaptive LMS filter, an SPS, a GRU, an LSTM, or an RNN.
17. The method of any of clauses 12 to 16, wherein analyzing the object based on the data includes determining a time-of-flight between the single ultrasound transducer and the object.
18. The method of clause 17, wherein determining the time-of-flight includes performing a cross-correlation method or a minimum threshold method on the data.
19. The method of any of clauses 12 to 18, wherein analyzing the object based on the data includes at least one of: determining a velocity of the object; determining a distance between the single ultrasound transducer and the object; determining a size of the object; determining a thickness of the object; determining a mechanical characteristic of the object; or identifying the object.
20. The method of any of clauses 12 to 19, further including transmitting, to an external device, a communication signal indicative of the object.
21. The method of any of clauses 12 to 20, wherein removing the ringdown artifact includes applying at least one ML-based filter to the data.
22. The method of clause 21, further including: training the at least one ML-based filter using training data, wherein the training data includes example ultrasound signals generated in a controlled environment.
23. The method of any of clauses 12 to 22, wherein removing the ringdown artifact includes applying a bandpass filter, and wherein analyzing the object based on the data includes determining a time-of-flight between the single ultrasound transducer using a cross-correlation method.
24. The method of any of clauses 12 to 23, wherein removing the ringdown artifact includes applying an SPS, and wherein analyzing the object based on the data includes determining a time-of-flight between the single ultrasound transducer using a cross-correlation method.
25. The method of any of clauses 12 to 24, wherein removing the ringdown artifact includes applying an RNN, and wherein analyzing the object based on the data includes determining a time-of-flight between the single ultrasound transducer using a cross-correlation method.
26. The method of any of clauses 12 to 25, wherein analyzing the object based on the data includes applying at least one ML-based classifier to identify the object.
27. The method of clause 26, further including: training the at least one ML-based classifier using training data, wherein the training data includes data based on example objects including at least one of the object or one or more similar objects.
28. The method of any of clauses 12 to 27, further including providing a feedback signal indicative of the object wherein the feedback signal includes at least one of a visual signal, a tactile signal, or an audio signal.
29. The method of any of clauses 12 to 28, further including at least one additional sensor, wherein analyzing the object is further based on data from the at least one additional sensor.

### CONCLUSION

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be used for realizing implementations of the disclosure in diverse forms thereof.

Furthermore, numerous references have been made to patents, printed publications, journal articles and other written text throughout this specification. All such references are incorporated by reference herein in their entirety.

As will be understood by one of ordinary skill in the art, each implementation disclosed herein can comprise, consist essentially of or consist of its particular stated element, step, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the implementation to the specified elements, steps, ingredients or components and to those that do not materially affect the implementation. As used herein, the term “based on” is equivalent to “based at least partly on,” unless otherwise specified.

Unless otherwise indicated, all numbers expressing quantities, properties, conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing implementations (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or example of a language (e.g., “such as”) provided herein is intended merely to better illuminate implementations of the disclosure and does not pose a limitation on the scope of the disclosure. No language in the specification should be construed as indicating any non-claimed element essential to the practice of implementations of the disclosure.

Groupings of alternative elements or implementations disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

What is claimed is:

1. A laparoscopic instrument, comprising:
a probe;
an output device configured to provide a feedback signal; and
a single ultrasound transducer disposed at an end of the probe and configured to:
transmit an incident ultrasound signal; and
detect a received ultrasound signal at least partially from an object within a range of about 0.5 centimeters to about two centimeters of the single ultrasound transducer; and
at least one processor configured to:
remove, from data indicative of the received ultrasound signal, a ringdown artifact;
based on removing the ringdown artifact, determine, based on the data, a time-of-flight between the single ultrasound transducer and the object; and
cause the output device to provide the feedback signal based at least in part on the time-of-flight.

2. The laparoscopic instrument of claim 1, wherein removing the ringdown artifact comprises applying a recurrent neural network (RNN) to the data indicative of the received ultrasound signal; and
wherein determining, based on the data, the time-of-flight between the single ultrasound transducer and the object comprises estimating the time-of-flight by performing cross-correlation on the data.

3. The laparoscopic instrument of claim 1, wherein the output device comprises at least one of a light, a display, a haptic feedback device, or a speaker; and
wherein the feedback signal comprises at least one of a visual signal, a tactile signal, or an audio signal.

4. At least one device, comprising:
a single ultrasound transducer configured to:
transmit an incident ultrasound signal; and
detect a received ultrasound signal at least partially from a structure within a ringdown range the single ultrasound transducer; and
at least one processor configured to:
remove, from data indicative of the received ultrasound signal, a ringdown artifact; and
based on removing the ringdown artifact, determine, by analyzing the data, a distance to the structure or a characteristic of the structure.

5. The at least one device of claim 4, wherein a frequency of the incident ultrasound signal is in a range of about 2.5 MHz to about 3.5 MHz.

6. The at least one device of claim 4, wherein the structure comprises at least one of a soft tissue, a bone, a foreign body, or a tumor.

7. The at least one device of claim 4, wherein the ringdown artifact comprises a residual vibration of the single ultrasound transducer in response to transmitting the incident ultrasound signal.

8. The at least one device of claim 4, wherein removing the ringdown artifact comprises applying, to the data indicative of the received ultrasound signal, at least one of:
a bandpass filter,
an adaptive LMS filter,
an SPS,
a GRU,
an LSTM, or
an RNN.

9. The at least one device of claim 4, wherein the characteristic of the structure comprises at least one of:
a velocity of blood in the structure;
a velocity of a wall of the structure;
a size of the structure;
a thickness of the structure;
a mechanical characteristic of the structure; or
an identification of the structure.

10. The at least one device of claim 9, wherein analyzing the data comprises:
in response to removing the ringdown artifact from the data indicative of the received ultrasound signal, determining a frequency shift and/or a phase shift of the received ultrasound signal with respect to the incident ultrasound signal by analyzing the data; and
determining a velocity of a fluid in the structure based on the frequency shift and/or the phase shift.

11. The at least one device of claim 4, further comprising an output device configured to output an indication of the characteristic,
wherein the output device comprises at least one of a light source, a display, a speaker, or a haptic feedback device.

12. A method, comprising:
transmitting, by a single ultrasound transducer, an incident ultrasound signal;
detecting, at least partially from an object within a ringdown range of the single ultrasound transducer and by the single ultrasound transducer, a received ultrasound signal;
removing, from data indicative of the received ultrasound signal, a ringdown artifact; and
based on removing the ringdown artifact, analyzing the object based on the data.

13. The method of claim 12, wherein a frequency of the incident ultrasound signal is in a range of about 2.5 MHz to about 3.5 MHz.

14. The method of claim 12, wherein the object comprises a soft tissue, a bone, a foreign body, or a tumor.

15. The method of claim 12, wherein the ringdown artifact comprises a residual vibration of the single ultrasound transducer in response to transmitting the incident ultrasound signal.

16. The method of claim 12, wherein removing the ringdown artifact comprises applying, to the data indicative of the received ultrasound signal, at least one of:
a bandpass filter,
an adaptive LMS filter,
an SPS,
a GRU,
an LSTM, or
an RNN.

17. The method of claim 12, wherein analyzing the object based on the data comprises determining a time-of-flight between the single ultrasound transducer and the object.

18. The method of claim 17, wherein determining the time-of-flight comprises performing a cross-correlation method or a minimum threshold method on the data.

19. The method of claim 12, wherein analyzing the object based on the data comprises at least one of:
determining a velocity of the object;
determining a distance between the single ultrasound transducer and the object;
determining a size of the object;
determining a thickness of the object;
determining a mechanical characteristic of the object; or identifying the object.

20. The method of claim 12, further comprising transmitting, to an external device, a communication signal indicative of the object.
